(19) United States
(12) Patent Application Publication
(10) Pub. No.: US 2005/0131473 A1
(43) Pub. Date: Jun. 16, 2005

(54) Electrophysiology catheter workstation and cardiac stimulator apparatus and method

(75) Inventors: Pat L. Gordon, Austin, TX (US); David Jenkins, Flanders, NJ (US)

Correspondence Address: FITCH EVEN TABIN AND FLANNERY, 120 SOUTH LA SALLE STREET, SUITE 1600, CHICAGO, IL, 60603-3406, US

(73) Assignee: EP MedSystems, Inc.

(21) Appl. No.: 10/733,114

(22) Filed: Dec. 11, 2003

(51) Int. Cl. A61N 1/362

(52) U.S. Cl. 607/11

(57) ABSTRACT

An integrated electrophysicology catheter workstation and cardiac stimulator having a cardiac electrical waveform response monitor (11) having at least two modes of operation. During a first mode of operation the monitor receives, detects, processes, stores, and displays EKG data in an ordinary fashion. During a second mode of operation one or more components and/or signal pathways are automatically modified to essentially de-sense part or all of system to thereby prevent a cardiac stimulation pulse from degrading the operability of the monitor. This, in turn, permits the monitor to utilize the first mode of operation more rapidly following application of a stimulation pulse and thereby glean useful post-pulse EKG data.

### TECHNICAL FIELD

This invention relates generally to electrophysiology catheter laboratories and electrocardiograms.

### BACKGROUND

Cardiac electrophysiology catheter systems are known in the art. Such systems typically serve in a sterile (or semi-sterile) setting where electrode catheters are placed percutaneous through blood vessels of a sedated patient into or around the heart. This arrangement then serves to permit diagnosis and/or therapy for various cardiac electrical conduction concerns. Such systems often include an X-ray fluoroscopy machine, a cardiac stimulator (also often known as a pulse generator), and a computer workstation. A physician uses the fluoroscopy machine to place the electrode catheters. The cardiac stimulator sends electrical pulses through electrode catheters to excite the tissue of the heart and thereby, for example, provoke an arrhythmia or pace the heart in a programmed manner to thereby terminate an arrhythmia. The computer workstation serves to record the resultant cardiac electrical waveforms (i.e., the electrocardiograms (EKGs)) and to permit other corresponding processing including amplification, filtering, storage, and display of the waveforms.

Though useful to facilitate many diagnostic and therapeutic processes, such systems are nevertheless not wholly satisfactory for all potentially relevant purposes.

For example, such systems typically use a constant current-based cardiac stimulator. A typical implantable cardiac pacemaker, however, derives its pulse from a constant voltage source. Therefore, when performing a cardiac pacemaker implant procedure in a typical electrophysiology suite, a separate constant voltage pacemaker system analyzer must typically be utilized. Such an analyzer comprises a discrete component and consequently the electrophysiology equipment cannot afford a seamless interface that will readily permit, for example, automatic recording of the pacemaker system analyzer output parameters.

As another example, existing electrophysiology systems do not assure accurate recordation of resultant EKG's under all circumstances. For example, during the period of time shortly following application of a stimulating pulse by a cardiac stimulator, the recording capability (and/or other detection or processing capabilities) of the workstation can be significantly temporarily impaired due, at least in part, to the transient strength of the stimulating pulse. This window of time, however, can be important. For example, in the case of a train of stimulating pulses as constitutes a response to an induced tachycardia, an ability to obtain and have access to an undistorted EKG that represents the time essentially immediately following each individual pulse (including, for example, the first 50 milliseconds) can be critical. Such information can allow, for example, a clinician or automatic recognition software to determine whether a given stimulating pulse has indeed stimulated tissue or has occurred in a refractory interval.

Such problems are meant to be illustrative only. Again, though of relatively long-standing nature and use, existing electrophysiology systems are often partially or wholly unsuitable to meet the needs of various processes involving controlled stimulation and monitoring of the tissues of the heart.

### BRIEF DESCRIPTION OF THE DRAWINGS

The above needs are at least partially met through provision of the electrophysiology catheter workstation and cardiac stimulator apparatus and method described in the following detailed description, particularly when studied in conjunction with the drawings, wherein:

FIG. 1 comprises a block diagram as configured in accordance with various embodiments of the invention;

FIG. 2 comprises a block diagram as configured in accordance with various embodiments the invention;

FIG. 3 comprises a block diagram as configured in accordance with various embodiments of the invention;

FIG. 4 comprises a flow diagram as configured in accordance with various embodiments of the invention;

FIG. 5 comprises a block diagram as configured in accordance with various embodiments of the invention;

FIG. 6 comprises a detail block diagram and schematic as configured in accordance with an embodiment of the invention;

FIG. 7 comprises a detail block diagram and schematic as configured in accordance with an embodiment of the invention;

FIG. 8 comprises a block diagram and schematic as configured in accordance with an embodiment of the invention;

FIG. 9 comprises a series of timing diagrams as configured in accordance with an embodiment of the invention;

FIG. 10 comprises a series of timing diagrams as configured in accordance with another embodiment of the invention; and

FIG. 11 comprises an illustrative display as configured in accordance with various embodiments of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are typically not depicted in order to facilitate a less obstructed view of these various embodiments of the present invention.

### DETAILED DESCRIPTION

Generally speaking, pursuant to these various embodiments, an apparatus can comprise a cardiac stimulator, a cardiac electrical waveform recorder that comprises at least a first controllable cardiac electrical waveform path, and a controller that is operably responsive to the cardiac stimulator and that has a control signal output that is operably coupled to the first controllable cardiac electrical waveform path such that the controller can modify the first controllable cardiac electrical waveform path as a function, at least in part, of the cardiac stimulator. For example, pursuant to various illustrative embodiments, the first controllable cardiac electrical waveform path can comprise any of a sample and hold circuit, a cardiac electrical waveform amplifier, and an analog to digital converter, to name a few.

Pursuant to a preferred embodiment, the cardiac stimulator can include a cardiac stimulation pulse precursor signal output and the controller can be configured to be operably responsive to the cardiac stimulation pulse precursor signal output. In particular, this precursor signal can be used to vary the operation of the first controllable cardiac electrical waveform path to mitigate the impact of a cardiac stimulation pulse on the detection, recording, and/or processing capability of the waveform recorder.

So configured, the waveform recorder can be selectively desensitized to the occurrence of a stimulation pulse. In a preferred approach, ordinary operation of the waveform recorder resumes very rapidly following such a pulse. By protecting the waveform recorder from the transitory effects of the stimulation pulse, and by rapidly restoring ordinary operation of the waveform recorder following such a pulse, the waveform recorder can detect and respond in an ordinary and appropriate fashion to the response of the pulsed heart tissue during a time period essentially immediately following the pulse. This, in turn, permits accurate observation of phenomena having potentially important diagnostic value in many instances.

Pursuant to one embodiment, the cardiac stimulator can comprise a controlled voltage based cardiac stimulator. Pursuant to another embodiment, the cardiac electrical waveform recorder can also be configured to be responsive, at least in part, to a controlled current based cardiac stimulator. In a preferred approach, the apparatus includes both a controlled voltage based cardiac stimulator and a controlled current based cardiac stimulator. So configured, the apparatus can support, in an integrated fashion, procedures that rely upon a controlled voltage based approach and procedures that rely upon a controlled current based approach. This in turn yields any number of benefits including improved efficiencies, resultant accuracy, and support for procedures that might otherwise be postponed or eschewed due to lack of native support for one approach or the other.

Other embodiments are consistent with these various teachings as well as will be shown in more detail herein. As one example, a master clock can be shared amongst many or all of these varied components.

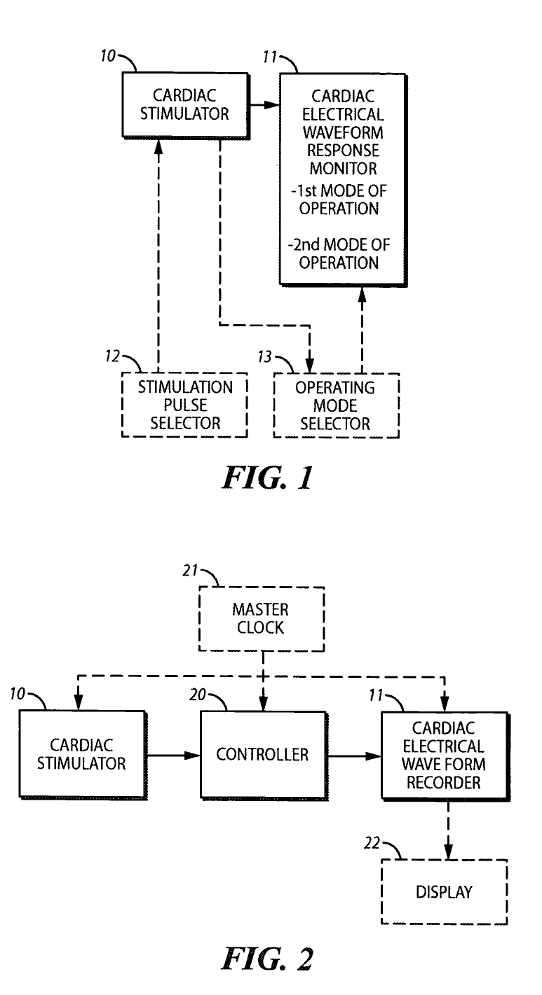

Referring now to the drawings, and in particular to FIG. 1, an integrated electrophysiology catheter workstation and cardiac stimulator having substantially uninterrupted electrocardiogram recording capability can be comprised generally of a cardiac stimulator 10 and a cardiac electrical waveform response monitor 11. In a preferred embodiment the cardiac stimulator 10 has an output that provides cardiac stimulation pulses. That cardiac stimulator output operably couples to the cardiac electrical waveform response monitor 11. In one embodiment, the output of the cardiac stimulator provides a biphasic cardiac stimulation pulse. Pursuant to these embodiments, the output of the cardiac stimulator 10 can be either of a controlled current stimulation pulse (including but not limited to a constant current stimulation pulse) and a controlled voltage stimulation pulse (including but not limited to a constant voltage stimulation pulse).

In a preferred embodiment the cardiac electrical waveform response monitor 11 has at least a first mode of operation and a second mode of operation. Viewed generally, pursuant to the first mode of operation the cardiac electrical waveform response monitor processes cardiac electrical waveform response information from the cardiac stimulator 10 using a first process and pursuant to the second mode of operation the cardiac electrical waveform response monitor processes the cardiac electrical waveform response information using a second process, which second process is different from the first process. Pursuant to a preferred approach, one of these processes will comprise a substantially normal process wherein the cardiac electrical waveform response information is detected and processed in accordance with ordinary processing while the other process comprises a de-sensed mode of operation for the cardiac electrical waveform response monitor 11, such that the monitor 11 will be less sensitive to the cardiac stimulation pulse. The latter process can be used, for example, to ensure that the former process, when used, will tend to yield valid and accurate results notwithstanding temporal proximity of a cardiac stimulation pulse.

As noted above, the cardiac stimulator 10 can comprise either of a controlled voltage or a controlled current based platform. Pursuant to one embodiment, the cardiac stimulator 10 can facilitate either approach. So configured, the apparatus will also then preferably include a stimulation pulse selector 12 such that provision of the controlled current stimulation pulse or the controlled voltage stimulation pulse is responsive to the stimulation pulse selector 12. Such a selector 12 can be provided in any of a wide variety of ways, including but not limited to graspable or otherwise manipulable buttons, switches, levers, knobs, or other control surfaces, a touch-sensitive display, a keypad, a speech recognizer, and so forth.

The apparatus can also optionally include an operating mode selector 13. The latter will preferably be operably responsive to an operational state (either present or anticipated) of the cardiac stimulator 10. So configured, the operating mode selector 13 can select a particular operating mode for use by the cardiac electrical waveform response monitor 11 as a function of an operating state of the cardiac stimulator 10. For example, the operating mode selector 13 can select a particular mode of operation (such as a de-sensed mode of operation) for use during an event window that includes provision of a cardiac stimulation pulse (where, for example, such an event window precedes by at least some period of time provision of the cardiac stimulation pulse). Similarly, the operating mode selector 13 can select another mode of operation (such as a normal mode of operation) for use during times other than during such an event window. As will be described below in more detail, such operational behavior can be effected in one embodiment by having the operating mode selector 13 detect a precursor signal that provides an early indicia of the imminent provision of a cardiac stimulation pulse and use such detection to initiate the event window.

Referring now to FIG. 2, a controller 20 can be interposed between the cardiac stimulator 10 and the cardiac electrical waveform recorder 11 (or physically incorporated into one or the other) to facilitate the above-described activity. In such an embodiment, the cardiac waveform recorder 11 preferably includes at least a first controllable cardiac electrical waveform path (such as, but not limited to, a sample and hold circuit, a cardiac electrical waveform amplifier, and/or an analog to digital converter). The controller 20 is preferably operably responsive to the cardiac stimulator 10 and has a control signal output that operably couples to the controllable cardiac electrical waveform path such that the controller 20 can modify the controllable cardiac electrical waveform path (and hence the operability of the cardiac electrical waveform recorder 11) as a function, at least in part, of the cardiac stimulator 10). In a preferred embodiment, the controller 20 particularly responds to a cardiac stimulation pulse precursor signal output as sourced by the cardiac stimulator 10.

Pursuant to one embodiment the apparatus further includes a master clock 21. So configured, the master clock 21 can serve as a primary clock source for one or more of these components, including but not limited to the cardiac stimulator 10, the controller 20, and the cardiac electrical waveform recorder 11 as illustrated. Such a configuration permits both heightened integration and further may aid in achieving improved synchronicity of executed behavior and functionality as between these components.

Pursuant to another embodiment the apparatus includes a display 22. This display 22 can comprise any suitable display as meets the needs of a given set of operational requirements and can include, for example, a cathode ray tube display, a liquid crystal display (or other pixelated display platform), a projection display, and so forth. Such a display 22 can operably couple to the cardiac electrical waveform recorder 11 and can serve to display information that corresponds to detected cardiac electrical waveform responses. For example, the displayed information can describe, at least in part, a given cardiac stimulation pulse (including information that describes a cardiac stimulation pulse using generated information as based upon previously stored information in a manner to be described in more detail below).

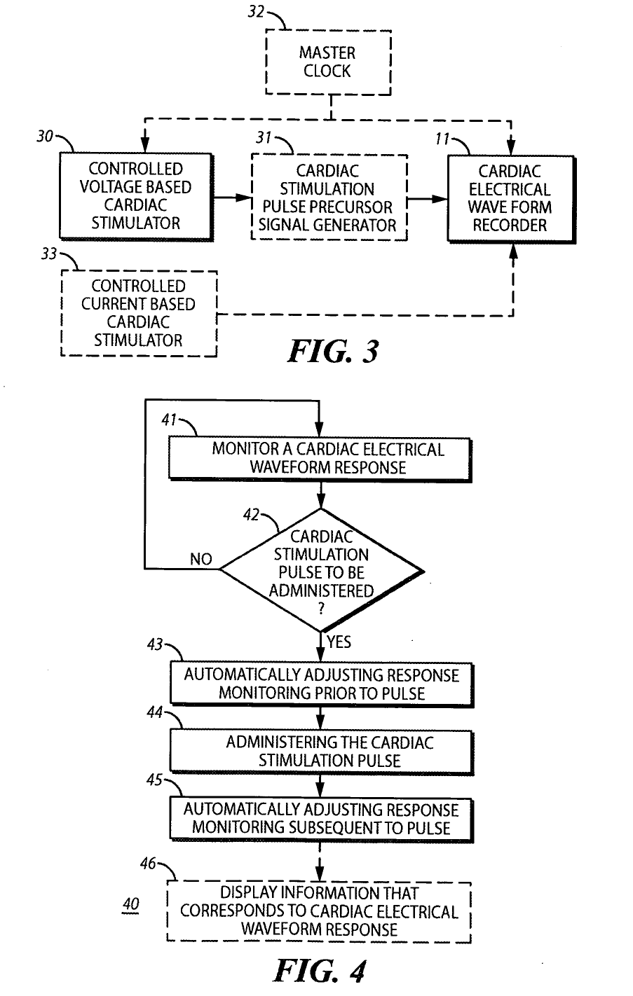

Referring now to FIG. 3, pursuant to certain embodiments, a cardiac stimulator, such as a controlled voltage based cardiac stimulator 30 can operably couple to a cardiac electrical waveform recorder 11 via, for example, an optional cardiac stimulation pulse precursor signal generator 31. As illustrated the cardiac stimulation pulse precursor signal generator 31 has a presence independent of the cardiac stimulator 30. If desired, of course, these two components can be configured integral to one another. In a preferred embodiment, the output of the cardiac stimulator 30 comprises a biphasic cardiac stimulation pulse. More particularly, and still pursuant to a preferred approach, the biphasic cardiac stimulation pulse has an initial portion that is characterized by a positive waveform and a trailing portion that is characterized by a negative waveform. More particularly still, and still pursuant to a preferred approach, this trailing portion of the biphasic cardiac stimulation pulse can have a duration that corresponds, at least in part, to a comparison between a present value of the negative waveform and a previously stored value (wherein, for example, the previously stored value corresponds, at least in part, to a voltage across the electrodes of the cardiac stimulator 30 prior to provision of a cardiac stimulator pulse).

So configured, the cardiac stimulation pulse precursor signal generator 31 can be responsive to the controlled voltage based cardiac stimulator 30 so as to permit provision of a corresponding cardiac stimulation pulse precursor signal output to the cardiac electrical waveform recorder 11. Such a precursor signal will preferably be provided at least a predetermined period of time prior to administration of the corresponding cardiac stimulation pulse. Such a precursor signal can be utilized as described above to permit selective alteration of the operation of the recorder 11 to avoid undue disruptions to the operations of the recorder 11. For example, the cardiac electrical waveform recorder 11 can have a processor (comprising, for example, at least one of an analogue signal processing element and a digital signal processing element) wherein the processor is suitably responsive to such a stimulation pulse precursor signal.

Such a processor and/or any other suitable platform can respond to such a precursor signal, for example, by essentially shielding the cardiac electrical waveform recorder from stimulator pulses as may be sourced by the controlled voltage based cardiac stimulator 30. So configured, for example, the apparatus can selectively control the impedance across the electrodes of a cardiac stimulator 30 subsequent to provision of a cardiac stimulator pulse being provided by the cardiac stimulator 30 (for example, by temporarily reducing this impedance). This in turn can facilitate the display and storage of cardiac electrical waveforms by the cardiac electrical waveform recorder 11 during at least an initial 100 millisecond period following such a cardiac stimulation pulse wherein the cardiac electrical waveform is substantially free of distortion and artifacts due to the cardiac stimulation pulse. Such a capability constitutes a significant improvement and can provide vitally useful information regarding certain conditions of the heart.

As mentioned above, a master clock 32 can be utilized to synchronize the activities of, for example, the controlled voltage based cardiac stimulator 30 and the cardiac electrical waveform recorder 11. This master clock 32 can provide clock signals to other elements and components as desired. As also mentioned above, a controlled current based cardiac stimulator 33 can be provided in addition to the controlled voltage based cardiac stimulator 30 as desired and/or as appropriate to the needs of a given application. Such a controlled current based cardiac stimulator 33 can operably couple to the cardiac electrical waveform recorder 11, either relatively directly as illustrated or through a (or the) cardiac stimulation pulse precursor signal generator 31.

These various embodiments can serve to facilitate a process 40 as generally set forth at FIG. 4. This process 40 provides for the monitoring 41 of a cardiac electrical waveform response and the determination 42 of when a cardiac stimulation pulse is to be administered. For example, monitoring decisions can be based upon the provision and/or detection of a precursor signal as described above. Upon determining that a cardiac stimulation pulse is to be administered, the process 40 automatically adjusts 43 the monitoring of the cardiac electrical waveform response prior to administration of the cardiac stimulation pulse. For example, in a preferred approach, the monitoring process will be adjusted within about 0.1 to 30 milliseconds of administering the cardiac stimulation pulse. The general purpose of this modification is to effect a diminution of detection and/or response capability with respect to administration of the cardiac stimulation pulse.

Pursuant to one embodiment, the modification can comprise substantially halting conversion of analog information that corresponds to sensed cardiac activity into a digital representation thereof. As an optional variation, at least one interpolated cardiac electrical waveform response value can be employed such that this interpolated value is used to substitute for the lack of a real-time pulse activity counterpart. To illustrate, an interpolated value that corresponds to a graceful transition between the pre-pulse waveform and the post-pulse waveform can be utilized during the time the process 40 has halted the conversion of cardiac activity analog information into corresponding digital content.

Pursuant to another embodiment, the modification can comprise temporarily substantially de-coupling a value that corresponds to a sensed value of a sensed cardiac electrical response from the sensed cardiac electrical response. For example, the sensed value can be substantially maintained at a given stored value (such as a present value as corresponds to measured phenomena regarding the cardiac electrical waveform response as measured across the electrocardiogram electrodes at the time of effecting the adjusted response) regardless of later variations to the cardiac electrical response as may occur during some subsequent period of time.

Other adjustment techniques are suitable for use as well, either alone or in combination with adjustment techniques such as those presented above. For example, the gain of the pertinent cardiac electrical waveform response signal path can be reduced (or fully attenuated) to facilitate a desired de-sensing of the cardiac electrical waveform recorder 11 to the impact of a cardiac stimulation pulse event.

The process 40 then administers 44 the anticipated biphasic cardiac stimulation pulse. In a preferred embodiment, this stimulation pulse will stimulate heart tissue using an electrode and will administer a pulse sufficient to discharge (preferably completely) the interface capacitance between the electrode and the tissue. As noted earlier, this pulse will preferably have an initial portion characterized by a positive polarity and a subsequent portion (such as a trailing portion) characterized by an opposite negative polarity. Such a trailing portion can comprise, for example, a trailing edge ramp waveform. As will be shown below in more detail, such a trailing portion can be effectively utilized to support the intent of these embodiments.

Subsequent to administration 44 of the stimulation pulse, the process 40 automatically adjusts 45 the response monitoring. This can occur at a predetermined period of time after some predetermined trigger point (such as the initial automatic adjustment 43 of the response monitoring capability of the apparatus) or can comprise a dynamically determined period of time as appropriate to the needs and requirements of a given application. This automatic adjustment 45 can be calibrated, for example, to occur within 20 milliseconds, or 10 milliseconds, of when the cardiac stimulation pulse concludes (or is expected to have concluded). In general, this adjustment 45 serves to return the monitoring capability of the apparatus to a normal mode of functionality. In other words, the apparatus recovers from the de-sensing the process 40 occasioned during the earlier automatic adjustment 43 of the monitoring response such that subsequent monitoring will equate with the monitoring capability as existed prior to the earlier automatic adjustment 43.

Optionally, the process 40 can display 46 information that corresponds to the cardiac electrical waveform response. This information can include information that describes, at least in part, the cardiac stimulation pulse (wherein, for example, this may include generating such information using previously stored information or otherwise interpolating or providing information to substitute for actual readings).

So configured, it will be readily appreciated that such embodiments, though varied, all serve to protect the monitoring and processing capabilities of the cardiac electrical waveform recorder 11 from the impact of a cardiac stimulation pulse. This, in turn, permits the recorder 11 to be available to accurately monitor the response of the heart tissue immediately subsequent to the administration of such a pulse in contrast to the capabilities of at least most prior art offerings. In addition, some of these embodiments permit selection between a controlled voltage based cardiac stimulator and a controlled current based cardiac stimulator. This, in turn, provides a high degree of integrated flexibility to better meet the varied needs of a given medical procedures suite or facility. Another benefit of these embodiments is that a more complicated (and hence diagnostically or therapeutically interesting) stimulation pulse shape can be applied (such as a biphasic pulse) while still remaining essentially assured of accurate and useful data capture.

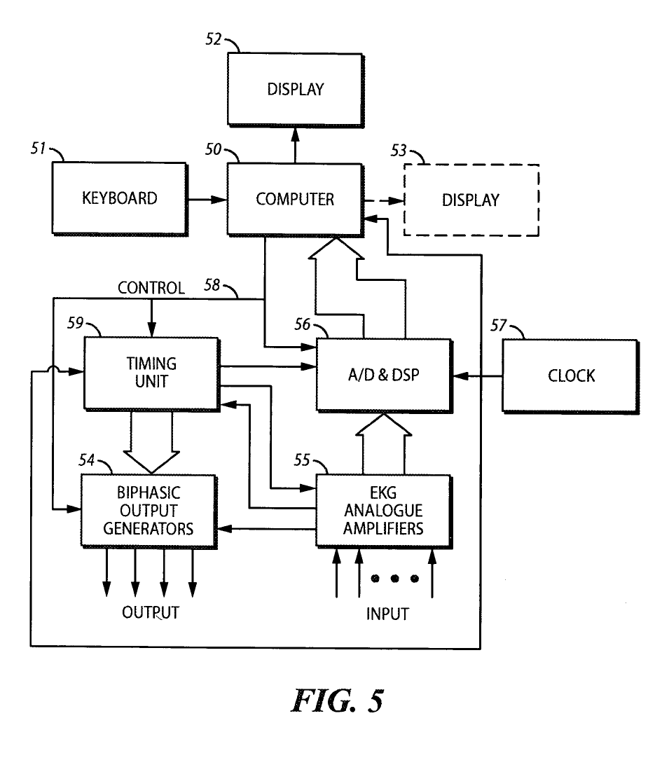

Referring now to FIG. 5, a more detailed example of a unified and integrated system will be described. In accordance with the teachings set forth above, this system will facilitate the capture, display, and storage of EKG signals that are substantially free of stimulator artifacts and corresponding distortion even during the 100 millisecond aftermath period that follows a stimulation pulse. In this embodiment these benefits are attained by activation of one or more of a sequence of signal path operations in the 10 to 20 millisecond period just prior to application of a stimulation pulse.

In this illustrative embodiment, the system comprises a computer 50 having a corresponding keyboard 51 or other user input mechanism and a display 52 (or displays 53—multiple displays may be desired during certain procedures to provide various participants a ready and unobstructed view of EKG data). The computer 50 is programmed to control (or even effect) in an integrated fashion the functioning of both the cardiac electrical waveform response monitor and the cardiac stimulator (the latter comprising, in this embodiment, a plurality of biphasic output generators 54) and hence can be viewed as an integral element of both.

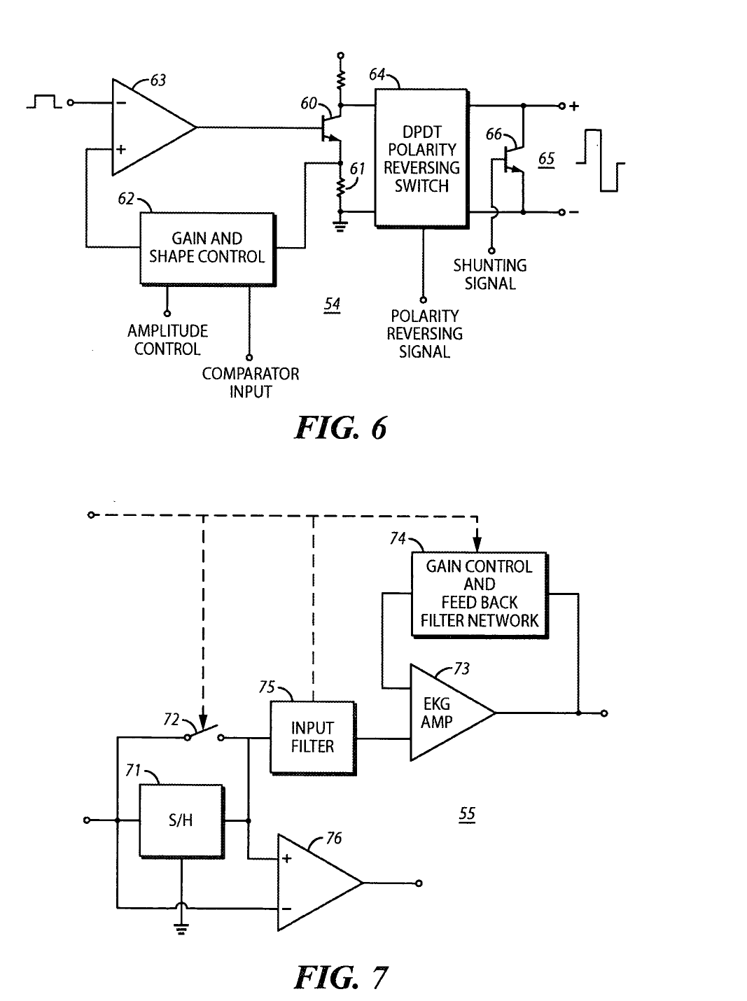

Referring momentarily to FIG. 6, the biphasic output generators 54 are preferably configured as illustrated and include an output switch 60, a current sensing resistor 61, a feedback gain and shape control unit 62, and an operational amplifier 63. The operational amplifier 63 will preferably receive a digital signal of constant amplitude (such as, for example, a constant current signal in the range of 1.0 to 20 milliamps) as generated by the timing unit 59 (FIG. 5). The feedback unit 62 responds to amplitude control instructions from the computer 50 (FIG. 5) and regulates the resultant amplitude accordingly. This feedback unit 62 also responds to a comparator input as related in more detail below.

Following delivery of a pulse to the input of the biphasic output generator 54, a double pole double throw polarity reversing switch responds to a polarity reversing signal as sourced, in this embodiment, by the timing unit 59 (FIG. 5) and reverses the polarity connections between the switch 60 and the output of the biphasic output generator 54. This reversal of polarity results in the provision of a negative pulse. This negative pulse comprises a delivered charge substantially equal to the preceding pulse and therefore serves to actively discharge the interface capacitance between the electrodes and the stimulated heart tissue.

An impedance lowering shunting switch 66 also couples to these output electrodes 65 and responds to a shunting signal as described in more detail below.

A controllable cardiac electrical waveform path comprises, in this embodiment, EKG analogue amplifiers 55 that operably couple to receive sensed EKG information and an analog to digital converter and digital signal processor 56. During ordinary operation, each EKG analogue amplifier 55 receives and amplifies to a useful signal range the incoming EKG signals. In a preferred embodiment, and referring now momentarily to FIG. 7, the EKG analogue amplifiers 55 can be comprised of a sample and hold unit 71 that stores the analogue voltage value that is present across the output electrodes 65 (FIG. 6) of the biphasic output generators. An input switch 72 can be selectively opened and the gain of the EKG amplifier 73 can be lowered (for example, by selectively changing the feedback via a gain control and feedback filter network 74). As another option, the charge on capacitors included in an input filter 75 can be selectively held constant to effect a similar functionality.

So configured, the gain for the EKG amplifier 73 can be selectively reduced in response, for example, to delivery of a cardiac stimulation pulse. A reduced gain, in turn, will aid in preventing the stimulation pulse from overpowering and inappropriately de-sensing other downstream components and processing.

In this embodiment the EKG analogue amplifiers 55 also include a comparator 76 configured as shown (and having an output coupled to the comparator input of the gain and shape control 62 of the biphasic output generators 54 as described above with respect to FIG. 6). The purpose of this comparator 76 will be made clearer below.

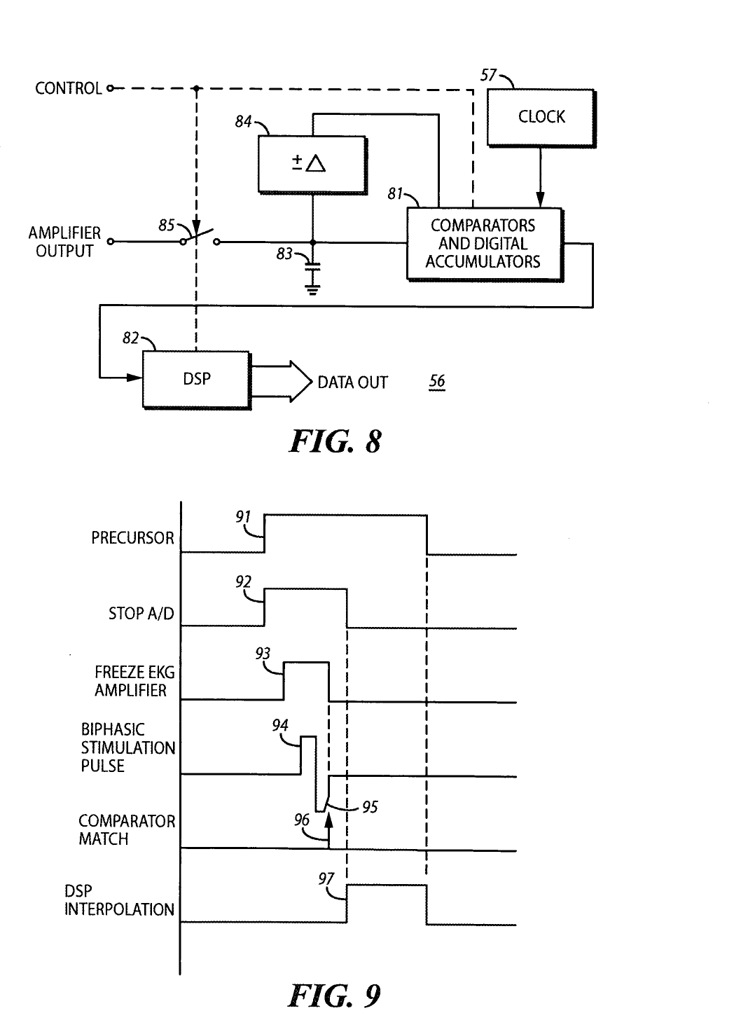

Referring again to FIG. 5, an analogue to digital converters/digital signal processor unit 56 has analogue to digital converters that convert amplified analogue information into a digital counterpart. The digital signal processor then processes this digitized EKG information as appropriate to a given application (for example, this EKG information may be filtered, additionally amplified, normalized, or otherwise processed as desired). With momentary reference to FIG. 8, this unit 56 is comprised in this embodiment of comparators and digital accumulators 81 that provide analog to digital conversion functionality and a digital signal processor 82. As will be shown below, these comparators and digital accumulators 81 are responsive to a control signal that causes a cessation of the conversion process. When this occurs, a so-called prediction analogue voltage is held constant on a capacitor 83 via the action of a differential modulator (such as a delta modulator, a differential pulse code modulator, a sigma delta modulator, or the like). Such control signals can also cause the digital accumulators to remain fixed in value and/or to open an overload protection switch 85.

When such control signals are provided (during, for example, provision of a cardiac stimulation pulse), the digital signal processor 82 can respond in a variety of ways depending upon the embodiment selected. For example, pursuant to one embodiment, the digital signal processor 82 will fill-in the signal corresponding to a time when the analog to digital converters are frozen. Pursuant to a preferred approach, the digital signal processor 82 can output an interpolated EKG value(s) to replace the missing analog to digital conversion samples. Digital signal processors typically require some amount of time to effect their processing. As a result, some amount of time delay may be expected when effecting such an interpolation function. As one optional approach, the digital signal processor 82 can compute derivatives of the incoming signals and compute a spline function that can be used to file in any signal portions that might otherwise be missing due to such hysterisis to thereby reduce associated distortion.

Referring again to FIG. 5, it can be seen that, pursuant to these embodiments, these various elements of the controllable cardiac electrical waveform path can be automatically altered to thereby de-sense the path to the effects of a stimulation pulse. This in turn permits the path to be viable immediately following such a pulse.

In this embodiment the components comprising the system operate synchronously pursuant to use of a common clock 57. The computer 50 then sources timing commands via a control bus 58 to the analog to digital converters/digital signal processor 56, the biphasic output generators 54, and a stimulator timing unit 59. These commands can include real-time commands or instructions comprising a precursor notification to the analog to digital converters/digital signal processor 56 to modify their processing in anticipation of a stimulation pulse. Other commands can include a command or instruction to the stimulator timing unit 59 to cause the creation of a digital pulse train to be provided to the biphasic output generators 54. Yet another command to the stimulator timing unit 59 can comprise blanking signal commands that the timing unit 59 can respond to by providing digital control signals to the EKG analogue amplifiers 55 to alter the configuration and/or functionality or behavior thereof before, during, and/or immediately following a cardiac stimulation pulse. Yet another command can include a command to the timing unit 59 to cause the latter to create a real time digital control signal to be provided to the analog to digital converters/digital signal processor 56 to cause a direct change to the operability thereof as otherwise set forth above.

So configured, such a system can provide the desired de-sensing using any or all of the above indicated approaches. These actions, in turn, de-sense the monitoring and recording capabilities of the system to thereby facilitate provision of a real time monitoring and recording capability that is fully functional immediately following application of a cardiac stimulation pulse.

FIG. 9 provides a series of timing diagrams that generally depict a relative time relationship regarding various operations as pertain to such embodiments. A precursor signal 91 begins prior to delivery of a cardiac stimulation pulse as described above and can be provided, for example, by the computer 50 and/or the timing unit 59 or such other component as can serve this purpose in a given implementation. The duration of the precursor signal 91 should preferably be co-extensive with the various other actions that are described below.

In response to receiving (or sourcing) the precursor signal, in this embodiment the timing unit 59 and/or the computer 50 sources a signal 92 to stop the conversion activities of the analog to digital converters of the comparators and digital accumulators 81 of the analog to digital converters and digital signal processor 56. This stop signal 92, in this embodiment, has a duration that is sufficient to include the stimulation pulse but that is not so long as to occlude significant portions of the aftermath response. This duration can be of fixed length or can be rendered dynamic and responsive to other conditions and instructions regarding its duration. Depending upon the embodiment, this same signal 92 can also be used to hold constant the prediction analogue voltage on the capacitor 83 of the analog to digital convertor and digital signal processor 56, to hold the digital accumulators 81 (FIG. 8) fixed in value, and/or to open the overload protection switch 85 (FIG. 8) described above. All of these actions tend to protect one or more elements of the analog to digital converters and digital signal processor 56 and/or other downstream processing elements from harm and/or distorted data due to the intensity of the anticipated cardiac stimulation pulse.

In a somewhat similar fashion, another signal 93 can be provided to the EKG analogue amplifiers 55. This signal 93 can serve to use the sample and hold unit 71 to essentially freeze the output value provided by this unit as a present value. This signal 93 can also be used to influence the gain of the EKG analogue amplifiers 55 and/or to influence the input filters 75 (FIG. 7) thereof. Again, these actions tend to protect downstream processing elements from harm and/or distorted data that may be occasioned by the intensity of a cardiac stimulation pulse.

The biphasic output generators 54 then apply a biphasic stimulation pulse 94. As already noted, this pulse 94 has a positive polarity initial portion and a trailing portion that has a negative polarity. In a preferred embodiment the shape control unit 62 (FIG. 6) serves in part to shape the trailing portion of the stimulation pulse as a trailing edge ramp waveform 95. In this embodiment, the duration of the stimulation pulse 94 is dynamically determined using the comparator 76 (FIG. 7) provided with the EKG analogue amplifiers 55. When the amplitude of the incoming signal matches a previous value as stored by the sample and hold unit 71, the comparator 76 provides a signal 96 to the biphasic output generators 54 to cause termination of the stimulation pulse. This comparator signal 96 can also be provided to the timing unit 59 (or, optionally, to the computer 50) to cause, for example, subsequent control signaling to unfreeze the functioning of the EKG analogue amplifiers 55.

The digital signal processor 56 can then be signaled to effect interpolation 97 of that portion of the incoming signal as corresponds to that period of time when the stop signal 92 was applied to the analog to digital converters.

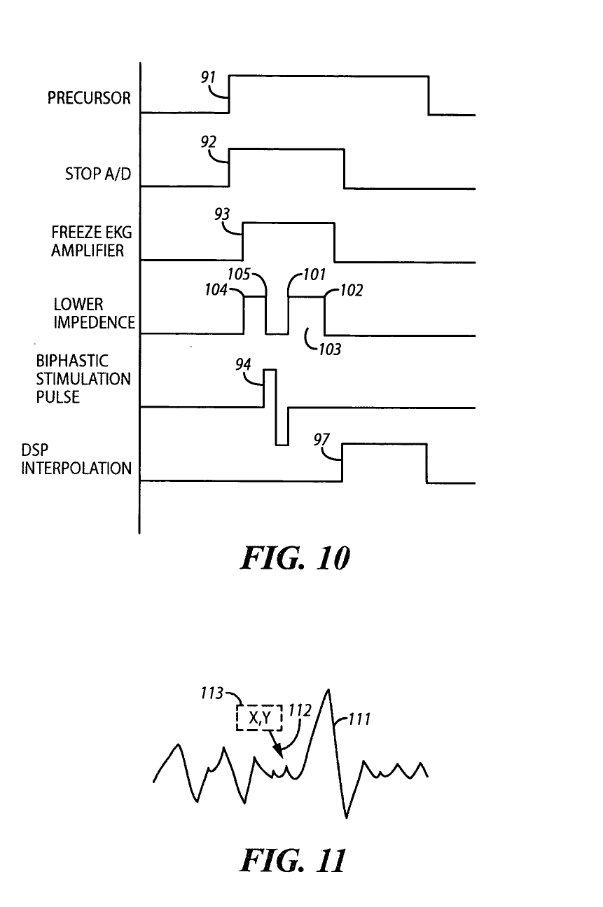

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept. For example, and referring now to FIG. 10, one alternative embodiment does not use a comparator. Instead, the negative portion of the biphasic stimulation pulse 94 is equal in duration and amplitude to the positive portion thereof. After the initiation of the stimulation pulse 94, the shunting switch 66 (FIG. 6) of the biphasic output generators 54 closes between a first time 101 and a second time 102 (to define a window of time of, for example, 10 milliseconds) to thereby lower the impedance between the stimulating electrodes to (typically) less than 500 ohms and preferably from 10 to 100 ohms and to substantially discharge the inter-electrode potential. In addition to this period 103 of reduced impedance, if desired, the shunting switch 66 can also be closed at a first time 104 before the stimulation pulse (such as 10 milliseconds prior to the onset of the stimulation pulse) and a second time 105 after the pulse has begun.

As another example, and referring now to FIG. 11, a resultant EKG display (such as a hardcopy printout and/or an electronically displayed representation) can include both a depiction of an EKG response 111 and one or more other graphic elements 112 that correspond to the administration of a stimulation pulse. For example, such a graphic element 112 can generally or specifically identify a time at which the stimulation pulse was administered with respect to the EKG response. Such an integration can serve both to shorten the amount of time a technician requires to analyze and interpret such results and to contribute to greater accuracy with respect to such interpretation as well. If desired, the stimulation pulse graphic element(s) 112 can comprise one or more graphic icons, expressions, or other depictions of choice and can further include, as appropriate, other annotations and information 113 that correspond to, for example, one or more characteristics of the stimulation pulse. To illustrate, such annotations 113 could include the pulse width of the stimulation pulse, the amplitude of the stimulation pulse, and any other information regarding the stimulation pulse that may be helpful to archive or that may otherwise inform the analysis process.

1. An apparatus comprising:
a control-voltage based cardiac stimulator;
a cardiac electrical waveform recorder that is responsive, at least in part, to the control-voltage based cardiac stimulator.

2. The apparatus of claim 1 and further comprising a control-current based cardiac stimulator, wherein the cardiac electrical waveform recorder is also responsive, at least in part, to the control-current based cardiac stimulator.

3. The apparatus of claim 1 and further comprising a cardiac stimulation pulse precursor signal generator that is responsive to the control-voltage cardiac stimulator and having a cardiac stimulation pulse precursor signal output that is operably coupled to the cardiac electrical waveform recorder.

4. The apparatus of claim 3 wherein the cardiac electrical waveform recorder comprises a processor that comprises at least one of:
at least one analogue signal processing element; and
at least one digital signal processing element;
wherein the processor is operably responsive to the cardiac stimulation pulse precursor signal output.

5. The apparatus of claim 1 and further comprising a master clock and wherein the control-voltage based cardiac stimulator and the cardiac electrical waveform recorder are both responsive to the master clock.

6. The apparatus of claim 1 and further comprising control means for shielding the cardiac electrical waveform recorder from control-voltage based cardiac stimulator pulses.

7. The apparatus of claim 6 wherein the control means facilitates at least one of the display and storage of cardiac electrical waveforms, during at least an initial 100 millisecond period following a cardiac stimulation pulse, wherein the cardiac electrical waveform is substantially free of distortion and artifacts due to the cardiac stimulation pulse.

8. The apparatus of claim 6 wherein the control means temporarily reduces impedance across electrodes of a cardiac stimulator subsequent to provision of a cardiac stimulator pulse being provided by the cardiac stimulator.

9. The apparatus of claim 6 wherein the control-voltage based cardiac stimulator has an output that provides a biphasic cardiac stimulation pulse.

10. The apparatus of claim 9 wherein the biphasic cardiac stimulation pulse has an initial portion that is characterized by a positive waveform.

11. The apparatus of claim 10 wherein the biphasic cardiac stimulation pulse has a trailing portion that is characterized by a negative waveform.

12. The apparatus of claim 11 wherein the trailing portion has a duration that corresponds, at least in part, to a comparison between a present value of the negative waveform and a previously stored value.

13. The apparatus of claim 12 wherein the previously stored value corresponds, at least in part, to a voltage across electrodes of a cardiac stimulator prior to provision of the cardiac stimulator pulse.

14. An apparatus comprising:
a cardiac stimulator;
a cardiac electrical waveform recorder comprising at least a first controllable cardiac electrical waveform path;
a controller that is operably responsive to the cardiac stimulator and having a control signal output operably coupled to the first controllable cardiac electrical waveform path such that the controller can modify the first controllable cardiac electrical waveform path as a function, at least in part, of the cardiac stimulator.

15. The apparatus of claim 14 wherein the cardiac stimulator comprises at least one of:
a control-voltage based cardiac stimulator; and
a control-current based cardiac stimulator.

16. The apparatus of claim 14 wherein the first controllable cardiac electrical waveform path comprises at least one of:
a sample and hold circuit;
a cardiac electrical waveform amplifier; and
an analog to digital converter.

17. The apparatus of claim 14 wherein the cardiac stimulator provides a biphasic cardiac stimulation pulse.

18. The apparatus of claim 14 wherein the cardiac stimulator comprises a cardiac stimulation pulse precursor signal output and wherein the controller is operably responsive to the cardiac stimulation pulse precursor signal output.

19. The apparatus of claim 14 and further comprising a master clock and wherein the cardiac stimulator and the cardiac electrical waveform recorder both use the master clock as a primary clock source.

20. The apparatus of claim 19 wherein the controller also uses the master clock as a primary clock source.

21. A method comprising:
monitoring a cardiac electrical waveform response;
determining that a cardiac stimulation pulse is to be administered;
automatically adjusting the monitoring of the cardiac electrical waveform response prior to administration of the cardiac stimulation pulse;
administering the cardiac stimulation pulse;
automatically adjusting the monitoring of the cardiac electrical waveform response subsequent to administration of the cardiac stimulation pulse.

22. The method of claim 21 wherein determining that a cardiac stimulation pulse is to be administered comprises providing at least a first precursor signal at least a predetermined period of time prior to administering a corresponding cardiac stimulation pulse.

23. The method of claim 21 wherein automatically adjusting the monitoring of the cardiac electrical waveform response prior to administration of the cardiac stimulation pulse comprises modifying the monitoring of the cardiac electrical waveform response to thereby diminish detection of the cardiac stimulation pulse when administered.

24. The method of claim 23 wherein modifying the monitoring of the cardiac electrical waveform response to thereby diminish detection of the cardiac stimulation pulse when administered comprises substantially halting conversion of analog information into a digital representation thereof.

25. The method of claim 24 wherein substantially halting conversion of analog information into a digital representation thereof comprises substantially halting conversion of analog information that corresponds to sensed cardiac activity into a digital representation thereof.

26. The method of claim 23 wherein modifying the monitoring of the cardiac electrical waveform response to thereby diminish detection of the cardiac stimulation pulse when administered comprises temporarily substantially de-coupling a value that corresponds to a sensed value of a sensed cardiac electrical response from the sensed cardiac electrical response.

27. The method of claim 26 wherein comprises temporarily substantially de-coupling a value that corresponds to a sensed value of a sensed cardiac electrical response from the sensed cardiac electrical response comprises substantially maintaining the sensed value at a present value regardless of subsequent variations to the cardiac electrical response that occur within some period of time subsequent to maintaining the sensed value at the present value.

28. The method of claim 27 wherein the some period of time comprises a predetermined period of time.

29. The method of claim 27 wherein the some period of time comprises a dynamically determined period of time.

30. The method of claim 23 wherein modifying the monitoring of the cardiac electrical waveform response to thereby diminish detection of the cardiac stimulation pulse when administered comprises storing a present value as corresponds to measured phenomena regarding the cardiac electrical waveform response.

31. The method of claim 30 wherein storing a present value as corresponds to measured phenomena regarding the cardiac electrical waveform response comprises storing a present value as corresponds to measured phenomena regarding the cardiac electrical waveform response as measured across electrocardiogram electrodes.

32. The method of claim 31 wherein storing a present value as corresponds to measured phenomena regarding the cardiac electrical waveform response as measured across electrocardiogram electrodes comprises storing a value that corresponds to a present voltage across the electrocardiogram electrodes.

33. The method of claim 23 wherein modifying the monitoring of the cardiac electrical waveform response to thereby diminish detection of the cardiac stimulation pulse when administered comprises altering gain as pertains to a cardiac electrical waveform response signal path.

34. The method of claim 33 wherein altering gain as pertains to a cardiac electrical waveform response signal path comprises reducing the gain.

35. The method of claim 21 wherein administering the cardiac stimulation pulse comprises administering a biphasic cardiac stimulation pulse.

36. The method of claim 21 wherein administering the cardiac stimulation pulse comprises administering a cardiac stimulation pulse having an initial portion comprised of a waveform having a first polarity.

37. The method of claim 36 wherein administering a cardiac stimulation pulse having an initial portion comprised of a waveform having a first polarity comprises administering a cardiac stimulation pulse having an initial portion comprised of a waveform having a positive polarity.

38. The method of claim 36 wherein administering a cardiac stimulation pulse having an initial portion comprised of a waveform having a first polarity further comprises administering a cardiac stimulation pulse having a portion subsequent to the initial portion comprised of a waveform having a second polarity that is opposite to the first polarity.

39. The method of claim 38 wherein the first polarity comprises a positive polarity and the second polarity comprises a negative polarity.

40. The method of claim 38 wherein administering a cardiac stimulation pulse having a portion subsequent to the initial portion comprised of a waveform having a second polarity that is opposite to the first polarity comprises administering a cardiac stimulation pulse having a portion subsequent to the initial portion comprised of a waveform having at least a portion thereof comprising a trailing edge ramp waveform.

41. The method of claim 38 wherein administering a cardiac stimulation pulse having a portion subsequent to the initial portion comprised of a waveform having a second polarity that is opposite to the first polarity further comprises:
determining when the portion subsequent to the initial portion has a value that corresponds in a predetermined way with respect to another parameter;
automatically concluding the cardiac stimulation pulse as a function, at least in part, of determining that the portion subsequent to the initial portion has a value that corresponds in the predetermined way with respect to the another parameter.

42. The method of claim 41 wherein determining when the portion subsequent to the initial portion has a value that corresponds in a predetermined way with respect to another parameter comprises determining when the portion subsequent to the initial portion has an amplitude that corresponds in a predetermined way with respect to another parameter.

43. The method of claim 41 wherein determining when the portion subsequent to the initial portion has a value that corresponds in a predetermined way with respect to another parameter comprises determining when the portion subsequent to the initial portion has a value that corresponds in a predetermined way with respect to a previous value that corresponds to a previously monitored cardiac electrical waveform response.

44. The method of claim 43 wherein determining when the portion subsequent to the initial portion has a value that corresponds in a predetermined way with respect to a previous value that corresponds to a previously monitored cardiac electrical waveform response comprises determining when the portion subsequent to the initial portion has a value that corresponds in a predetermined way with respect to a previous value that corresponds to a previously monitored voltage value across electrocardiogram electrodes.

45. The method of claim 41 wherein determining when the portion subsequent to the initial portion has a value that corresponds in a predetermined way with respect to another parameter comprises determining when the portion subsequent to the initial portion has a value that substantially equals another parameter.

46. The method of claim 21 wherein automatically adjusting the monitoring of the cardiac electrical waveform response subsequent to administration of the cardiac stimulation pulse comprises automatically adjusting the monitoring of the cardiac electrical waveform response subsequent to administration of the cardiac stimulation pulse within 20 milliseconds of when the cardiac stimulation pulse concludes.

47. The method of claim 46 wherein automatically adjusting the monitoring of the cardiac electrical waveform response subsequent to administration of the cardiac stimulation pulse within 20 milliseconds of when the cardiac stimulation pulse concludes comprises automatically adjusting the monitoring of the cardiac electrical waveform response subsequent to administration of the cardiac stimulation pulse within 10 milliseconds of when the cardiac stimulation pulse concludes.

48. The method of claim 21 wherein automatically adjusting the monitoring of the cardiac electrical waveform response subsequent to administration of the cardiac stimulation pulse comprises adjusting the monitoring to substantially equate with the monitoring of the cardiac electrical waveform response prior to the automatic adjustment of the monitoring of the cardiac electrical waveform response prior to administration of the cardiac stimulation pulse.

49. The method of claim 21 wherein automatically adjusting the monitoring of the cardiac electrical waveform response prior to administration of the cardiac stimulation pulse comprises using at least one interpolated cardiac electrical waveform response value.

50. The method of claim 21 wherein administering the cardiac stimulation pulse comprises administering a cardiac stimulation pulse that:
firstly stimulates a tissue using an electrode; and
secondly administers a pulse sufficient to discharge interface capacitance between the at least one electrode and the tissue.

51. The method of claim 50 wherein administering a pulse sufficient to discharge interface capacitance between the at least one electrode and the tissue continues until the interface capacitance is substantially fully discharged.

52. The method of claim 21 and further comprising displaying information that corresponds to the cardiac electrical waveform response.

53. The method of claim 52 wherein displaying information that corresponds to the cardiac electrical waveform response comprises further displaying information that describes, at least in part, the cardiac stimulation pulse.

54. The method of claim 53 wherein further displaying information that describes, at least in part, the cardiac stimulation pulse comprises generating at least part of the information using previously stored information.

55. The method of claim 52 and further comprising also displaying information that corresponds to the stimulation pulse.

56. The method of claim 55 wherein displaying information that corresponds to the stimulation pulse further comprises displaying a graphic indication of a temporal occurrence of a stimulation pulse with respect to the cardiac electrical waveform response.

57. The method of claim 52 wherein displaying information that corresponds to the stimulation pulse further comprises displaying information regarding a characteristic of the stimulation pulse.

58. The method of claim 57 wherein displaying information regarding a characteristic of the stimulation pulse further comprises displaying information regarding at least one of:
a pulse width of the stimulation pulse; and
a pulse amplitude of the stimulation pulse.

59. The method of claim 21 wherein:
monitoring the cardiac electrical waveform response;
determining that a cardiac stimulation pulse is to be administered;
automatically adjusting the monitoring of the cardiac electrical waveform response prior to administration of the cardiac stimulation pulse;
administering the cardiac stimulation pulse; and
automatically adjusting the monitoring of the cardiac electrical waveform response subsequent to administration of the cardiac stimulation pulse;
are all done synchronously.

60. The method of claim 59 and further comprising providing a master clock and wherein:
monitoring the cardiac electrical waveform response;
determining that a cardiac stimulation pulse is to be administered;
automatically adjusting the monitoring of the cardiac electrical waveform response prior to administration of the cardiac stimulation pulse;
administering the cardiac stimulation pulse; and
automatically adjusting the monitoring of the cardiac electrical waveform response subsequent to administration of the cardiac stimulation pulse;
are all done synchronously as a function, at least in part, of the master clock.

61. The method of claim 21 wherein automatically adjusting the monitoring of the cardiac electrical waveform response prior to administration of the cardiac stimulation pulse comprises automatically adjusting the monitoring of the cardiac electrical waveform response within about 0.1 to 30 milliseconds of administering the cardiac stimulation pulse.

62. An integrated electrophysiology catheter workstation and cardiac stimulator with substantially uninterrupted electrocardiogram recording capability comprising:
a cardiac stimulator having an output that provides cardiac stimulation pulses;
a cardiac electrical waveform response monitor responsive to the cardiac stimulation pulses and having at least a first mode of operation and a second mode of operation, wherein:
pursuant to the first mode of operation the cardiac electrical waveform response monitor processes cardiac electrical waveform response information using a first process; and
pursuant to the second mode of operation the cardiac electrical waveform response monitor processes cardiac electrical waveform response information using a second process, which second process is different from the first process.

63. The integrated electrophysiology catheter workstation and cardiac stimulator of claim 62 wherein the output of the cardiac stimulator provides a biphasic cardiac stimulation pulse.

64. The integrated electrophysiology catheter workstation and cardiac stimulator of claim 63 wherein the output of the cardiac stimulator provides one of a substantially constant current stimulation pulse and a substantially constant voltage stimulation pulse.

65. The integrated electrophysiology catheter workstation and cardiac stimulator of claim 64 wherein the cardiac stimulator includes a stimulation pulse selector and wherein the provision of one of a substantially constant current stimulation pulse and a substantially constant voltage stimulation pulse is responsive to the stimulation pulse selector.

66. The integrated electrophysiology catheter workstation and cardiac stimulator of claim 62 and further comprising an operating mode selector that is operably responsive to an operational state of the cardiac stimulator.

67. The integrated electrophysiology catheter workstation and cardiac stimulator of claim 66 wherein the operating mode selector further comprises operating mode selection means for selecting the first mode of operation during an event window that includes provision of the cardiac stimulation pulse.

68. The integrated electrophysiology catheter workstation and cardiac stimulator of claim 67 wherein the operating mode selection means are further for selecting the second mode of operation during at least some time other than during the event window.

69. The integrated electrophysiology catheter workstation and cardiac stimulator of claim 68 wherein the event window precedes by at least some period of time provision of the cardiac stimulation pulse.

70. The integrated electrophysiology catheter workstation and cardiac stimulator of claim 69 wherein the operating mode selection means initiates the event window in response to detecting a precursor signal that provides an early indicia of imminent provision of the cardiac stimulation pulse for initiating the event window.

71. The integrated electrophysiology catheter workstation and cardiac stimulator of claim 62 wherein the first mode of operation comprises a de-sensed mode of operation for the cardiac electrical waveform response monitor, such that the monitor will be less sensitive to at least one of detection and processing of the cardiac stimulation pulse.
